(19) United States
(12) Patent Application Publication
(10) Pub. No.: US 2015/0250723 A1
(43) Pub. Date: Sep. 10, 2015

(54) NON-PEGYLATED LIPOSOMAL DOXORUBICIN COMBINATIONS FOR THE TREATMENT OF TRIPLE NEGATIVE BREAST CANCER

(71) Applicant: SOPHERION THERAPEUTICS, LLC, East Brunswick, NJ (US)

(72) Inventors: Marcel Rozencweig, East Brunswick, NJ (US); Ronald H. Goldfarb, East Brunswick, NJ (US); Salvatore Forenza, East Brunswick, NJ (US)

(21) Appl. No.: 14/426,196

(22) PCT Filed: Sep. 5, 2013
(86) PCT No.: PCT/US13/58246

§ 371 Date: Mar. 5, 2015

Related U.S. Application Data
(60) Provisional application No. 61/697,110, filed on Sep. 5, 2012.

(51) Int. Cl. A61K 9/127; A61K 31/337; A61K 31/427; A61K 31/475; A61K 31/555; A61K 31/704; A61K 31/7068

(52) U.S. Cl. CPC A61K 9/127; A61K 31/704; A61K 31/337; A61K 31/7068; A61K 31/475; A61K 31/555; A61K 31/427

(57) ABSTRACT

The present invention relates to a method for treating triple-negative metastatic breast cancer in an individual comprising administering to an individual in need thereof a dosing regimen which comprises nonpegylated liposomal doxorubicin, a taxane and a chemotherapeutic agent selected from the group consisting of capecitabine, vinorelbine, gemcitahine, carboplatin, and ixahepilone, and wherein said individual previously has been administered an anthracycline. In some embodiments, the individual is not administered transtuzumab.

### CROSS-REFERENCE TO RELATED APPLICATIONS

This application claims priority to U.S. Provisional Application No. 61/697,110, filed Sep. 5, 2012, the contents of which are herein incorporated by reference in their entirety.

### FIELD OF THE INVENTION

The present invention is in the field of treatment for triple-negative metastatic breast cancer in an individual who has previously been administered an anthracycline.

### BACKGROUND OF THE INVENTION

Breast cancer is one of the most common causes of cancer deaths in women. In the United States, it accounts for 30% of all malignancies that affect women, excluding non-melanoma skin cancer. About one-third of newly diagnosed patients will eventually recur and/or develop metastatic disease.

Triple-negative breast cancer refers to any breast cancer that does not express the genes fix estrogen receptor (ER), progesterone receptor (PR) or Her2/neu, Triple-negative breast cancer accounts for approximately 15%-25% of all breast cancer cases. The overall proportion of triple-negative breast cancer is very similar in all age groups. Younger women have a higher rate of basal or BRCA related triple-negative breast cancer while older women have a higher proportion of apocrine, normal-like and rare subtypes including neuroendocrine triple-negative breast cancer. Among younger women African American and Hispanic women have a higher risk of triple-negative breast cancer, with African Americans facing worse prognosis than other ethnic groups.

The trouble with this type of breast cancer is that it is better characterized by what it lacks rather than mutations that it has, making it difficult to identify treatments specific for this disease. Triple-negative breast cancer is aggressive, has a high rate of metastases, and carries a poor prognosis.

Doxorubicin is one of the most active and versatile anticancer agents. It has an exceptionally broad spectrum of activity and plays a leading role in the curative and palliative therapy of a diverse group of malignancies, most notably breast cancer, lymphoma, soft tissue sarcoma, various pediatric malignancies, multiple myeloma, and advanced bladder cancer. However, doxorubicin is associated with serious and sometimes life threatening side effects. In particular, doxorubicins use is limited by the potential for patients to suffer irreversible cardiotoxicity. The incidence of clinically significant cardiomyopathy or congestive heart failure rises with increasing lifetime cumulative doses of doxorubicin. For this reason, most clinicians limit a patient's lifetime cumulative dose of doxorubicin to 450 mg/m²or less. Sub-clinical and occasionally overt cardiotoxicity may also occur at lower cumulative doses of doxorubicin, especially when the drug is given as part of a combination regimen that includes drugs such as cyclophosphamide or paclitaxel, as well as newer biologic therapies, including trastuzumab.

Clinically relevant cardiotoxicity has been a particular issue in combination regimens of doxorubicin and the taxanes, especially paclitaxel, and to a lesser extent docetaxel and albumin-bound paclitaxel (Abraxang). Three-fold greater cardiac toxicity was seen in women receiving the combination of trastuzumab and an anthracycline (either doxorubicin or epirubicin) and cyclophosphamide (AC), compared with the rates of cardiotoxicity that would have been expected with AC or trastuzumab alone.

Within the overall population of patients who were administered a dosing regimen of Myocet®, trastuzumab and paclitaxel, there was a sub-population of patients that previously were administered an anthracycline. With respect to the population of patients who were administered Myocet®; trastuzumab and paclitaxel, the dosing regimen did not substantially increase the risk of cardiotoxicity.

It is an object of the present invention to provide a method of treating triple-negative metastatic breast cancer in an individual who has previously ben administered an anthracycline.

### SUMMARY OF THE INVENTION

The present invention relates to a method for treating triple-negative metastatic breast cancer in an individual comprising administering to an individual in need thereof a dosing regimen which comprises administering to the individual nonpegylated liposomal doxorubicin and a taxane.

The present invention also provides a method for treating triple-negative metastatic breast cancer in an individual comprising administering to an individual in need thereof a dosing regimen which comprises administering to the individual nonpegylated liposomal doxorubicin, a taxane and a chemotherapeutic agent selected from the group consisting of capecitabine, vinorelbine, gemcitabine, carboplatin, and ixabepilone, and wherein said individual previously has been administered an anthracycline. In some embodiments, the individual is not administered trastuzumab.

### BRIEF DESCRIPTION OF THE FIGURES

FIG. 1 is a representation of a nonpegylated liposome doxorubicin.

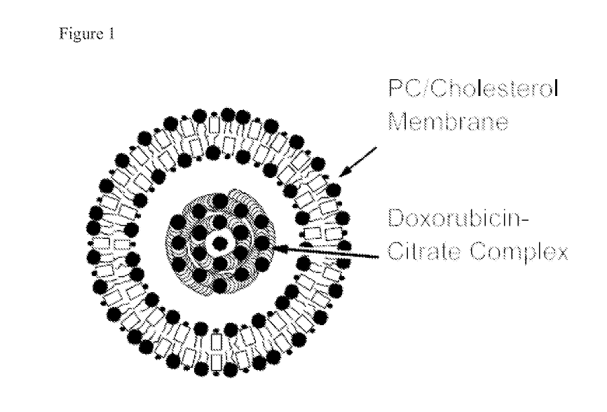

FIG. 2 shows the percent progression-free survival in HER2°/ER−PR− patients,

FIG. 3 shows the percent overall survival in HER2H-1ER−PR− patients.

### DETAILED DESCRIPTION OF THE INVENTION

The present invention provides a method for treating triple-negative metastatic breast cancer in an individual by administering to an individual in need thereof a dosing regimen which comprises administering to the individual nonpegylated liposomal doxorubicin and a taxane. Suitable taxanes which may be used in the present invention include paclitaxel, docetaxel and albumin-bound paclitaxel. In one embodiment the taxane is paclitaxel. In one such embodiment paclitaxel is administered once weekly. In another embodiment the taxane is docetaxel or albumin-bound paclitaxel. In another embodiment the taxane is docetaxel. In one such embodiment docetaxel is administered every 3 weeks. In another embodiment the taxane is albumin-bound paclitaxel. In one such embodiment albumin-bound paclitaxel is administered every 3 weeks.

The present invention also provides a method for treating triple-negative metastatic breast cancer in an individual by administering to an individual in need thereof a dosing regimen comprising nonpegylated liposomal doxorubicin, a taxane, and additional chemotherapeutic agents. Suitable additional chemotherapeutic agents include, without limitation, capecitabine, carboplatin, ixabepilone, gemcitabine, and vinorelbine. In some embodiments, the individual has previously been administered an anthracycline. In other embodiments, the individual is not administered trastuzumab.

In one embodiment of the present invention the dosing regimen does not substantially increase the likelihood that the individual will develop cardiotoxicity during or after the dosing regimen. In one such embodiment of the present invention the dosing regimen does not increase the likelihood that the individual will develop cardiotoxicity during or after the dosing regimen by more than 10%. In another such embodiment, the dosing regimen does not increase the likelihood that the individual will develop cardiotoxicity during or after the dosing regimen by more than 5%. In yet another such embodiment, the dosing regimen does not increase the likelihood that the individual will develop cardiotoxicity during or after the dosing regimen by more than 3%.

In one embodiment of the present invention the dosing regimen does not substantially increase the likelihood that the individual will develop a symptom of cardiotoxicity during or after the dosing regimen. In one such embodiment the symptom is reduced resting left ventricular ejection fraction.

In another embodiment of the present invention the dosing regimen does not substantially increase the likelihood that the individual will develop congestive heart failure during or after the dosing regimen. In one such embodiment of the present invention the dosing regimen does not increase the likelihood that the individual will develop congestive heart failure during or after the dosing regimen by more than 10%. In another such embodiment, the dosing regimen does not increase the likelihood that the individual will develop congestive heart failure during or after the dosing regimen by more than 5%. In yet another such embodiment, the dosing regimen does not increase the likelihood that the individual will develop congestive heart failure during or after the dosing regimen by more than 3%.

In one variation, the dosing regiment does not substantially increase the likelihood that the individual will develop New York Heart Association Class III or IV congestive heart failure during or after the dosing regimen. In one such embodiment of the present invention the dosing regimen does not increase the likelihood that the individual will develop New York Heart Association Class III or IV congestive heart failure during or after the dosing regimen by more than 10%. In another such embodiment, the dosing regimen does not increase the likelihood that the individual will develop New York Heart Association Class III or IV congestive heart failure during or after the dosing regimen by more than 5%. In yet another such embodiment, the dosing regimen does not increase the likelihood that the individual will develop New York Heart Association Class III or IV congestive heart failure during or after the dosing regimen by more than 3%.

In another embodiment of the present invention the dosing regimen does not substantially increase the likelihood that the individual will develop a symptom of congestive heart failure during or after the dosing regimen. In one such embodiment the symptom is dyspnea, tachycardia, cough, neck vein distention, cardiomegaly, hepatomegaly, paroxysmal nocturnal dyspnea, orthopnea or peripheral edema. In another such embodiment the symptom is dyspnea, tachycardia, neck vein distention, cardiomegaly, hepatomegaly, paroxysmal nocturnal dyspnea, orthopnea or peripheral edema.

In yet another embodiment of the present invention the dosing regimen does not substantially increase the likelihood that the individual will suffer cardiac death during or after the dosing regimen. In one such embodiment of the present invention the dosing regimen does not increase the likelihood that the individual will suffer cardiac death during or after the dosing regimen by more than 10%. In another such embodiment, the dosing regimen does not increase the likelihood that the individual will suffer cardiac death during or after the dosing regimen by more than 5%. In yet another such embodiment, the dosing regimen does not increase the likelihood that the individual will suffer cardiac death during or after the dosing regimen by more than 3%.

Elderly individuals have a higher rate of cardiac disease as well as chronic conditions, such as diabetes mellitus and hypertension, that may increase the risk of cardiac-related adverse events resulting from anthracycline administration. In one embodiment, the individual treated with a dosing regimen according to the present invention is at least 60 years of age. In one embodiment, the individual treated with a dosing regimen according to the present invention is at least 65 years of age. In one such embodiment, wherein the individual is at least 60 or at least 65 years of age, the individual has a condition that increases the risk of a cardiac-related adverse event resulting from anthracycline administration. In one embodiment the condition is diabetes or hypertension. In one such embodiment the condition is diabetes. In another such embodiment the condition is hypertension.

Palmar-plantar erythrodysesthesia, also known as hand-foot syndrome (HFS), is a side effect of certain chemotherapeutic agents that causes redness, swelling, and pain on the palms of the hands and/or the soles of the feet. HFS occurs when small amounts of chemotherapy leak out of the capillaries in the hands and feet. Once out of the blood vessels; the chemotherapy damages the surrounding tissues. Although less common, hand-foot syndrome can also occur on other areas of the skin, such as the knees and elbows.

The prescribing information provided with Doxil®, which is pegylated liposomal doxorubicin, reports that in the randomized ovarian cancer study, 50.6% of patients treated with Doxil at 50 mg/m² every 4 weeks experienced HFS (developed palmar-plantar skin eruptions characterized by swelling, pain, erythema and, for some patients, desquamation of the skin on the hands and the feet), with 23.8% of the patients reporting 1-IFS Grade 3 or 4 events, graded according to the NCI Common Toxicity Criteria Manual, Version 2.0 Jun. 1, 1999.

In another embodiment of the present invention the dosing regimen does not substantially increase the likelihood that the individual will develop palmar-plantar erythrodysesthesia during or after the dosing regimen. In one such embodiment, the dosing regimen does not increase the likelihood that the individual will develop palmar-plantar erythrodysesthesia during or after the dosing regimen by more than 10%. In another such embodiment, the dosing regimen does not increase the likelihood that the individual will develop palmar-plantar erythrodysesthesia during or after the dosing regimen by more than 8%. In yet another such embodiment, the dosing regimen does not increase the likelihood that the individual will develop palmar-plantar erythrodysesthesia during or after the dosing regimen by more than 6%. In a further ethbodiment, the dosing regimen does not increase the likelihood that the individual will develop Grade 3 or 4 events of palmar-plantar erythrodysesthesia during or after the dosing regimen by more than 5%. In one such embodiment, the dosing regimen does not increase the likelihood that the individual will develop Grade 3 or 4 events of palmar-plantar erythrodysesthesia during or after the dosing regimen by more than 4%. In yet another such embodiment, the dosing regimen does not increase the likelihood that the individual will develop Grade 3 or 4 events of palmar-plantar erythrodysesthesia during or after the dosing regimen by more than 3%. In yet another such embodiment, the dosing regimen does not increase the likelihood that the individual will develop Grade 3 or 4 events of palmar-plantar erythrodysesthesia during or after the dosing regimen by more than 2%.

In still another embodiment, the dosing regimen does not substantially increase the likelihood that the individual with develop either one of palmar-plantar erythrodysesthesia and congestive heart failure during or after the dosing regimen. In one such embodiment, wherein the dosing regimen comprises administering a chemotherapeutic agent selected from the group consisting of capecitabine, vinorelbine, gemcitabine, carboplatin, and ixabepilone, the dosing regimen does not, increase the likelihood that the individual with develop either one of palmar-plantar erythrodysesthesia and congestive heart failure during or after the dosing regimen by more than 5% over that which would be expected based upon administration of the given chemotherapeutic agent, alone.

As used herein, the term “triple-negative breast cancer” refers to any breast cancer that does not express the genes for estrogen receptor (ER), progesterone receptor (PR) Her2/neu.

As used herein, the term “concomitant” refers to events occur with 48 hours of each other.

As used herein, treatment that “does not substantially increase the likelihood” that the individual undergoing treatment will develop a particular condition, such as palmar-plantar erythrodysesthesia or congestive heart failure during such dosing regimen, refers to treatment that does not increase the probability that the individual undergoing treatment will develop the specified condition by more than 10%.

As used herein “previously administered” refers to administration of a drug, such as an anthracycline, at least 12 months before administration of the specified dosing regimen.

In one embodiment, the previous anthracycline administration is for the treatment of cancer. In one such embodiment, the previous anthracycline administration is for the treatment of breast cancer. The anthracycline previously administered to the individual may be any anthracycline that is approved for treatment in humans, such as, for example, doxorubicin, idarubicin, epirubicin, daunorubicin and valrubicin. Generally, the anthracycline previously administered to the individual is doxorubicin, idarubicin, epirubicin, or daunorubicin. In one embodiment, the anthracycline is doxorubicin. In one such embodiment, the doxorubicin previously administered to the individual is non-liposomal doxorubicin.

In one embodiment the previous anthracycline administration is adjuvant or neo-adjuvant administration of an anthracycline. In one such embodiment the previous anthracycline administration is adjuvant administration of an anthracycline. In another such embodiment the previous anthracycline administration is neo-adjuvant administration of an anthracycline.

In one embodiment the total amount of the anthracycline previously administered to said individual is less than or equal to 300 mg/m² of doxorubicin or 600 mg/m² of epirubicin. In one such embodiment the total amount of the anthracycline previously administered to said individual is from 7 mg/m² to 450 mg/m². In another such embodiment the total amount of the anthracycline previously administered to the individual is from 100 mm g² to 400 mg/m². In still another such embodiment the total amount of the anthracycline previously administered to the individual is from 100 mg/m² to 300 mg/m². In yet another such embodiment the total amount of the anthracycline previously administered to the individual is from 200 mg/m² to 300 mg/m².

The total amount of anthracycline previously administered to the individual may vary depending upon the particular anthracycline previously administered. For example, where the previously administered anthracycline is epirubicin, the total amount of anthracycline previously administered may be up to, for example, 600 mg/m². On the other hand, where the previously administered anthracycline is doxorubicin, the total amount of anthracycline previously administered typically will not exceed 300 mg/m².

In one embodiment, the previous administration of anthracycline was for the treatment of cancer. In a further embodiment, the previous administration of anthracycline was for the treatment of breast cancer. Typically, the previous administration of an anthracycline will have occurred at least 12 months prior to initiation of the dosing regimen of the present invention. In one such embodiment, the previous administration of an anthracycline will have occurred at least 18 months prior to initiation of the dosing regimen of the present invention. In another such embodiment, the previous administration of an anthracycline will have occurred at least 24 months prior to initiation of the dosing regimen of the present invention.

A colony stimulating factor may be administered to individuals requiring supportive therapy for myelosuppression. Accordingly, in one embodiment the individual is administered, in addition to nonpegylated liposomal doxorubicin, a taxane and an additional selected agent, a pharmaceutically effective amount of a colony stimulating factor. In one such embodiment the colony stimulating factor is a granulocyte colony stimulating factor, a macrophage colony stimulating factor or a granulocyte macrophage colony stimulating factor. In one such embodiment the colony stimulating factor is filgrastim, pegfilgrastim, sargramostim, lenograstim or molgramostim.

Chemotherapy-induced nausea and vomiting (CINV) is a common side-effect of chemotherapy treatment. An antiemetie agent may be administered to individuals requiring treatment for chemotherapy-induced nausea and/or vomiting. Accordingly, in one embodiment the individual is administered, in addition to nonpegylated liposonial doxorubicin, a taxane and an additional selected agent a pharmaceutically effective amount of an antiemetic agent. In one embodiment the antiemetic agent is a 5-HT3 receptor antagonist, a dopamine antagonist, an NK₁ receptor antagonist, a steroid or a cannabinoid. In one such embodiment the antiemetie agent is a 5-HT3 receptor antagonist. In one such embodiment the 5-HT3 receptor antagonist is dolasetron, granisetron, ondansetron, palonosetron or tropisetron. In another embodiment the antiemetic agent is a dopamine antagonist. In one such embodiment the dopamine antagonist is domperidone, droperidol, haloperidol, chiorpromazine, promethazine, prochlorperazine, raetoehlopramide or alizapride. In another embodiment the antiemetic agent is an NK₁ receptor antagonist. In one such embodiment the NK₁ receptor antagonist is aprepitant. In another embodiment the antiemetic agent is a cannabinoid. In one such embodiment the cannabinoid is cannabis, dronabinol, nabilone or sativex. In another embodiment the antiemetic agent is a steroid. In one such embodiment the steroid is dexamethasone.

Generally, nonpegylated liposomal doxorubicin is administered by infusion over 1 hour and is not given as a bolus injection. Doses of nonpegylated liposomal doxorubicin refer to the doxorubicin HCl content delivered in the nonpegylated liposomal doxorubicin injections.

The nonpegylated liposomal doxortibiein administered according to the present invention may be prepared according to the procedures described in U.S. Pat. No. 5,616,341, the entire disclosure of which is expressly incorporated by reference herein. One example of a nonpegylated liposomal doxorubicin is MYOCET®, a complex of doxorubicin-citrate encapsulated within the aqueous core of single lamellar liposomes that are composed of e phosphatidylcholine:cholesterol (55:45 mole:mole). Encapsulation of doxorubicin can be achieved via an active loading process, which utilizes a proton concentration gradient, wherein the solution inside the liposome has an acidic pH and the solution outside the liposome has a basic pH. Once the doxorubicin is encapsulated, the internal complex is a flexible assembly of doxorubicin monomers stacked into fibers that are cross-linked by citrate into a hexagonal array with a 35 .ANG. lattice repeat. The drug to lipid ratio of the encapsulated formulation is approximately 0.25:1 (wt:wt) and the pH is 6.5 to 8.5.

In one aspect, a method for treating triple-negative metastatic breast cancer is provided. In one embodiment, the method for treating triple-negative metastatic breast cancer comprises administering a dosing regimen to an individual in need thereof, wherein the dosing regimen comprises nonpegylated liposomal doxorubicin and paclitaxel. In a further embodiment, the dosing regimen further comprises at least one other agent selected from the group consisting of capecitabine, carboplatin, ixabepilone, vinorelbine, and gemcitibine. In another embodiment, the individual has previously been administered, an anthracycline. In yet another embodiment, the individual is not administered trastuzumab.

In one embodiment, the method comprises at least one treatment cycle. In a further embodiment, the method comprises 2, 3, 4, 5, 6, or more treatment cycles. In some embodiments, the treatment cycle is at least about 1 week long. In other embodiments, the treatment cycle is about 2, 3, 4, 5, 6, or more weeks long. In certain embodiments, the dosing regimen comprises at least one 3-week long treatment cycle. In further embodiments, the dosing regimen comprises 2, 3, 4, 5, 6, or more 3-week long treatment cycles. In still further embodiments, the dosing regimen comprises six consecutive 3-week long treatment cycles.

In one embodiment, the method for treating triple-negative metastatic breast cancer comprises administering to an individual in need thereof a dosing regimen which comprises nonpegylated liposomal doxorubicin at a dose level of from about 10 mg/m² to about 250 mg/m². In a further embodiment, the dose level of liposomal doxorubicin is from about 20 mg/m² to about 150 mg/m². In a still further embodiment, the dose level of liposomal doxorubicin is from about 30 mg/m² to about 75 mg/m². In a yet further embodiment, the dose level of liposomal doxorubicin is about 50 mg/m². In some embodiments, the liposomal doxorubicin is administered on day 1 of each treatment cycle.

In one embodiment, the method for treating triple-negative metastatic breast cancer comprises administering to an individual in need thereof a dosing regimen which comprises paclitaxel at a dose level of from about 5 mg/m² to about 500 mg/m². In a further embodiment, the dose level of paclitaxel is from about 25 mg/m² to about 400 mg/m². In a still further embodiment, the dose level of paclitaxel is from about 50 mg/m² to about 250 mg/m². In some embodiments, the paclitaxel is administered on day 1 of each treatment cycle. In other embodiments, the paclitaxel is administered on day 1 of the treatment cycle and weekly thereafter. In a further embodiment, paclitaxel is administered at a dose level of about 80 mg/m² on day 1 of the first treatment cycle and every week thereafter.

In one embodiment, the method for treating triple-negative metastatic breast cancer comprises administering to an individual in need thereof a dosing regimen which comprises at least one agent selected from the group consisting of capecitabine, carboplatin, ixabepilone, vinorelbine, and gemcitibine. In some embodiments, the at least one other agent is administered on day 1 of each treatment cycle. In some embodiments, the at least one agent selected from the group consisting of capecitabine, carboplatin, ixabepilone, vinorelbine, and gemcitibine is administered according to the FDA approved dosing guidelines for said agents) on day 1 of the first treatment cycle and subsequently according to the recommended schedule for the agent selected.

In one embodiment, the method fur treating triple-negative metastatic breast cancer comprises administering to an individual in need thereof a dosing regimen which comprises at least one 3-week long treatment cycle, and wherein said individual is administered nonpegylated liposomal doxorubicin at a dose level of from 30 mg/m² to 75 mg/m² on day 1 of each treatment cycle, paclitaxel at a dose level of from 50 mg/m² to 250 mg/m² on day 1 of each treatment cycle, and at least one other agent selected from the group consisting of capecitabine, carboplatin, ixabepilone, vinorelbine, and gemcitibine on day 1 of each treatment cycle; and wherein said individual previously has been administered an anthracycline.

In another embodiment, the method for treating triple-negative metastatic breast cancer comprises administering to an individual in need thereof a dosing regimen which comprises about 50 mg/m² nonpegylated liposomal doxorubicin on day 1 of each treatment cycle; about 80 mg/m² paclitaxel on day 1 of the first treatment cycle and every week thereafter, or about 75 mg/m² on day 1 of each treatment cycle, or from about 200 mg/m² to about 250 mg/m² on day 1 of each treatment cycle; and at least one additional agent(s) administered according to the FDA approved dosing guidelines for said agent(s) on day 1 of the first treatment cycle and subsequently according to the recommended schedule for the agent selected. In a further embodiment, the dosing regimen comprises six consecutive 3-week long treatment cycles.

### EXAMPLE 1

### Preparation of Single Use Vial

Vial #1 Doxorubicin HCl for Injection is provided in glass vials sealed with butyl rubber stoppers and aluminum flip-off seals which contains:

| | |
|---|---|
| Doxorubicin HCl, USP | 50 mg |
| Lactose, NF (hydrous) | 250 mg |

Vial #2 Liposomes for Injection is provided in 2 ml type I flint glass tubing vial with grey stoppers siliconized with dimethicone and a flip-off seal which contains:

| | | |
|---|---|---|
| Egg Phosphatidylcholine | 142.6 | mg |
| Cholesterol, NF | 57.4 | mg |
| Citrate Buffer (57.6 mg/mL) q.s. | 2 | mL |

Vial #3 Buffer for Injection is provided in 5 ml type I molded glass vials with grey stoppers siliconized with dimethicone and a flip-off seal contains:

| | | |
|---|---|---|
| Sodium Carbonate anhydrous, NF | 54.6 | mg |
| Water for Injection, USP q.s. | 3.1 | mL |

Each prepared vial of nonpegylated liposomal doxorubicin contains 50 mg of doxorubicin HCl, and each milliliter of nonpegylated liposomal doxorubicin contains:

| | |
|---|---|
| Doxorubicin HCl | 2.0 mg |
| Egg phosphatidylcholine | 5.4 mg |
| Cholesterol | 2.2 mg |
| Citric acid, monohydrate | 4.4 mg |
| Sodium carbonate | 2.2 mg |
| Lactose | 10.0 mg |
| Sodium Chloride Injection | 7.2 mg |

Cholesterol is controlled with an additional test for purity of not less than 95.0% by HPLC. Egg phosphatidylcholine is obtained by purifying egg yolk through extractions and chromatography. Doxorubicin HCl for Injection may contain around about 5 mg methylparaben, NF; in the nonpegylated liposomal doxorubicin, 0.2 mg methylparaben may be present. Generally the mean diameter of the liposomes is 100-230 nm.

During manufacture the product is sterilized using a 0.22 μm filter. Generally, the citric acid buffer is prepared, the pH adjusted with sodium hydroxide solution, and filtered through a 0.2 μm nominal filter into a reactor. The liposomes are clarified by filtration and subsequently filtered through a 0.22 μm filter and stored under nitrogen pressure at 2-8° C. before filling. The vials are sterilized using dry heat and the stoppers are autoclaved. The particle size distribution is measured as one part of the in-process controls.

In preparation of the buffer, sodium carbonate is added to water for injection, the solution is mixed and filtered through a 0.22 μm filter into sterile tank and stored at room temperature prior to filling.

Step 1. Set Up

A. Turn on water bath and allow water to equilibrate at 58° C. (55 to 60° C.).

B. Remove Liposomal Doxorubicin Injection carton from the refrigerator.

Step 2. Reconstitute Doxorubicin HCl for injection, USP (Vial No. 1)

A. Withdraw 20 mL sodium chloride injection 0.9%) and inject into each 50-mg vial of Doxorubicin HCl for Injection, USP intended for preparation (vial No. 1).

B. Shake well in the inverted position to ensure doxorubicin is fully dissolved.

Step 3. Heat in Water Bath

A. Heat the Doxorubicin HCl for Injection, USP (vial No. 1) in a water bath (55 to 60° C.) for 10 minutes (not to exceed 15 minutes). While heating, proceed to Step 4.

Step 4. Adjust pH of Liposomes

A. Withdraw 1.9 mL of Liposomes for Injection (vial No. 2).

B. Inject into Buffer for Injection (vial No. 3). Pressure buildup may require venting.

C. Shake well.

Step 5. Add Liposomes to Doxorubicin
A. Using syringe, withdraw the entire vial contents of pH-adjusted liposomes (vial No. 3).

B. Remove Doxorubicin FICI for Injection, USP (vial No. 1) from the water bath. SHAKE VIGOROUSLY. Then IMMEDIATELY (within 2 minutes) inject pH-adjusted liposomes into vial of heated 50 mg Doxorubicin HCl for Injection, USP (vial No. 1).

C. SHAKE VIGOROUSLY.

D. WAIT FOR A MINIMUM OF 10 MINUTES BEFORE USING.

### EXAMPLE 2

In a study conducted to evaluate the safety and efficacy of the combination of non-pegylated liposomal doxoruhicin (MYOCET®), paclitaxel and trastuzumab as first-line treatment in patients with HER2-over-expressing metastatic breast cancer, the inventors have determined that a key component of the trial patients population was HER2+ and ER−PR−. In this subpopulation, unexpectedly, a remarkable and statistically significant improvement of both progression-free survival (PFS) (6+ months) and overall survival (10+ months), with an overall Response duration of 5.6 months, was observed.

Inclusion and exclusion criteria for the study included subjects with HER2+ metastatic breast cancer by FISH analysis; no prior chemotherapy treatment for metastatic disease; no prior anthracyclines, taxanes, or trastuzumab within the 12 months prior to enrollment; no prior treatment with ≧300 mg/m² doxorubicin or with ≧600 mg/m² epirubicin; normal left ventricular ejection fraction; and no symptomatic brain metastases.

In treatment arm A (the Myocet group), subjects received 50 mg/m² Myocet® every 3 weeks for 6 cycles; 80 mg/m² paclitaxel weekly; and 4 mg/kg trastuzumab on day 1, then 2 mg/kg trastuzumab weekly. In treatment arm B (the control group), subjects received 80 mg/m² paclitaxel weekly; and 4 mg/kg trastuzumab on day 1, then 2 mg/kg trastuzumab weekly. Subjects were treated with paclitaxel and trastuzumab to progression or untoward toxicity.

Tumor assessments (thorax and abdominal scan) were conducted at baseline; every 3 days during treatment cycles; and every 3 months post-treatment until progression. Cardiac evaluations were conducted by multi-gated acquisition (MUGA) scans or echography at baseline and every two cycles for 24 weeks, then at 9, 12, 18, and 24 months.

The primary endpoint of the study was progression free survival (PFS). Progression was defined as radiographic progression per blinded central review, or death from any cause. Overall survival was also assessed. Patient accrual, demographics, and receptor status are shown below in Tables 1, 2, and 3, respectively.

TABLE 1
Accrual by Strata

| | Myocet | Control |
|---|---|---|
| | N = 181 | N = 182 |
| Age | | |
| >50 | 60% | 60% |
| ≦50 | 40% | 40% |
| Prior anthracyclines | | |
| Prior | 33% | 33% |
| No prior | 67% | 67% |
| Geography | | |
| North Am | 9% | 8% |
| Others | 91% | 92% |

TABLE 2
Demographics

| | Myocet | Control |
|---|---|---|
| | N = 181 | N = 182 |
| Age | | |
| Median | 52 | 53 |
| Range | 22-79 | 30-76 |
| Performance status (ECOG) | | |
| 0 | 62% | 61.5% |
| 1 | 38% | 38.5% |
| Prior adjuvant chemotherapy | | |
| Any chemotherapy | 42% | 38% |
| Anthracyclines | 33% | 33% |
| Taxanes | 8% | 7% |
| Trastuzumab | 1% | 2% |

TABLE 3
Receptor Status

| | Myocet | Control |
|---|---|---|
| | N = 181 | N = 182 |
| Hormone receptors | | |
| ER+, PR+ or both* | 41% | 45% |
| ER− and PR− | 49% | 48% |
| Unknown | 9% | 7% |
| HER2 + (FISH) | | |
| Positive | 98% | 98% |
| Negative | 2% | 2% |

*Includes borderline positivity

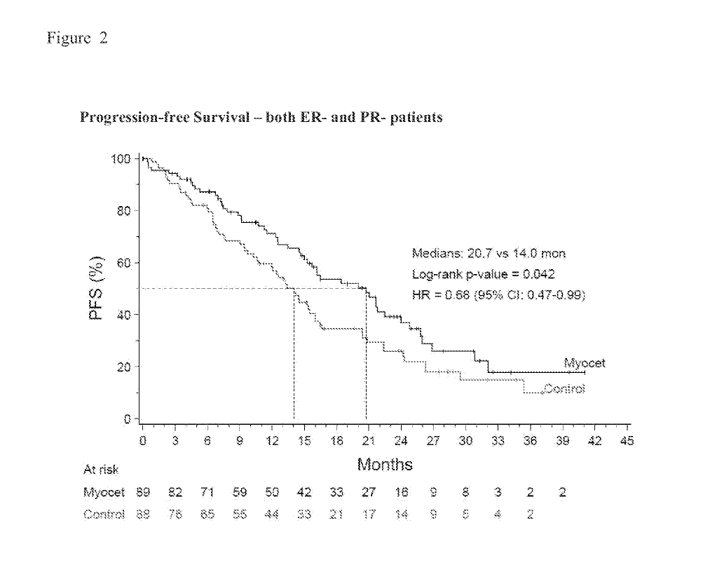

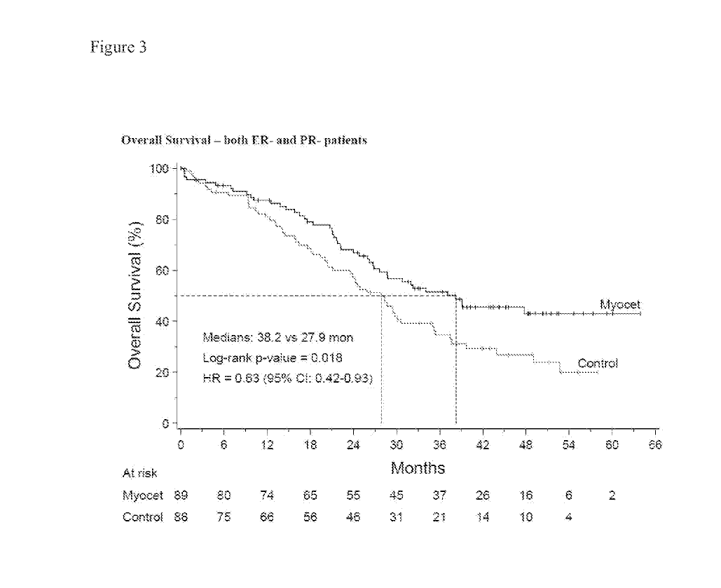

The progression-free survival (PFS) of patients that were both ER− and PR− is shown in FIG. 2. Percent PFS was significantly higher over time in the Myocet group as compared to the control group (log-rank p-value=0.042). The overall survival of patients that were both ER− and PR− is shown in FIG. 3. Percent overall survival was significantly higher in the Myocet group as compared to the control group (log-rank p-value=0.018).

Without wishing to be bound by theory, it is thought that the underlying unexpected positive results in the HER2+ and ER−PR− subpopulation are due to the sensitivity of the ER−/PR− patients to the combination of Myocet® and the taxane, since it has been demonstrated, both in clinical trials and oncology practices, that trastuzumab does not contribute any specific efficacy benefit to this population of metastatic breast cancer patients; the therapeutic role of trastuzumab is independent of the hormone receptor status.

Therefore, it is believed that the significantly improved PFS and, more importantly. Overall Survival observed are due to the combination of Myocet® and paclitaxel. The ER−/PR− tumor type sensitivity demonstrated in our trial is expected to benefit triple-negative patients in the absence of the HER2+ receptor-targeting trastuzumab.

### EXAMPLE 3

Based on the results of the study described above in Example 2, which was conducted to evaluate the safety and efficacy of the combination of non-pegylated liposomal doxorubicin (MYOCET®), paclitaxel and trastuzumab as first-line treatment in patients with HER2-over-expressing metastatic breast cancer, a study will be conducted to determine whether the combination of MYOCET® and paclitaxel or another taxane will show efficacy and clinical benefit in patients diagnosed with triple-negative metastatic breast cancer.

Combination with additional agents such as capecitabine, vinorelbine, gemcitahine, carboplatin, and ixabepilone may further enhance the positive outcome of treatment.

The proposed study to demonstrate the efficacy of such combinations has the following parameters.

Inclusion criteria for the study are: (1) triple-negative characterization of the metastatic disease; (2) no prior chemotherapy for metastatic disease; (3) measurable disease; and (4) normal left ventricular ejection fraction. Exclusion criteria are: (1) prior doxorubicin treatment exceeding 300 mg/m² or prior epirubicin treatment exceeding 600 mg/m²; and (2) relapse within 12 months of completion of adjuvant treatment or anthracycline therapy.

The primary objectives of this trial are to demonstrate the efficacy and cardiac safety of Myocet® when given in combination with paclitaxel and gemcitibine or capecitabine, or ixabepilone, or carboplatin, or ixabepilone, in patients with triple-negative metastatic breast cancer.

The trial will have the following design:

Primary Efficacy and Safety Endpoints:
Progression-Free Survival (PFS)
Overall Survival (OS)
New York Heart Association Class III & IV congestive heart failure and cardiac death.

Secondary Efficacy and Safety Endpoints:
Objective Response Rate
Safety Profile

Progression Free Survival (PFS) is defined as the time from randomization until the date of disease progression or death due to any cause. For the primary analysis, PFS events will be the PFS events as defined by a blinded independent review board.

The cardiac safety endpoint is defined as New York Heart. Association Class III or Class IV congestive heart failure and cardiac death. For the primary analysis, cardiac safety events will be the events as defined by a blinded independent review board.

Objective Response Rate is defined as the fraction of patients with complete or partial response as assessed by the Response Evaluation Criteria In Solid Tumors (RECIST). For the primary analysis, response will be the responses as defined by a blinded independent review board.

Overall Survival is defined as the time from randomization to the date of death.

The Safety Profile will compare treatment arms for worst grade adverse events; non-cardiac deaths and other serious adverse events; and worst grade laboratory abnormalities. Non-cardiac toxicity is assessed according to the National Cancer Institute (NCI) Common Terminology Criteria for Adverse Events (CTCAE) version 3.0.

Study Design, Randomization, Stratification

This is a Phase III, randomized, controlled, multi-center clinical trial in triple-negative patients with metastatic breast cancer. Myocet® will be administered (treatment arm A) at a dose of 50 mg/m² IV over one hour. Treatment cycles will be repeated every 3 weeks unless precluded by disease progression or unacceptable toxicity. Paclitaxel will be administered at a dose of 80 mg/m² over one hour weekly IV.

Dose escalation will not be permitted, however, one dose reduction of Myocet® or paclitaxel may be made for specific hematological and non-hematological toxicity,

Scheduling of the selected third agent (to be agreed upon with FDA) will be as per the characteristics of the agent and its delivery modalities

Colony Stimulating Factor (CSF) therapy may be administered as needed.

Patients will continue Myocet® treatment for a maximum of 6 cycles or until disease progression, the occurrence of unacceptable toxicity requiring discontinuation of study therapy, or for other reasons described in Criteria for Discontinuation, below.

Patients in both arms of the study will continue treatment on paclitaxel up to disease progression or until the occurrence of unacceptable toxicity requiring discontinuation of study therapy, or for other reasons described in Criteria for Discontinuation, below.

Criteria for Discontinuation

Study therapy is discontinued for any of the following:

1. Progressive disease.

2. Protocol-defined cardiotoxicity

3. Any grade 3 non-hematological toxicity that does not reverse to grade 1 or less within 35 days of study drug administration (except for alopecia). If the toxicity can be attributable to a specific drug, then that drug should be held rather than removing the patient from the study.

4. Any grade 4 non-hematological toxicity (with exception of fever or infection), regardless of reversibility. If the toxicity can be attributable to a specific drug, then that drug should be held rather than removing the patient from the study.

5. Grade 4 neutropenia, thrombocytopenia or anemia that does not reverse to grade 2 or less within 35 days of study drug administration.

6. Patient non-compliance or significant protocol deviation.

7. Pregnancy.

8. An intercurrent illness that in the opinion of the investigator, would prevent completion of study-related evaluations.

9. At the discretion of the investigator(s) and/or sponsor, or at the request of the patient.

Randomization

Arm A:
Myocet 50 mg/m.sup.2 every 3 weeks
Paclitaxel 80 mg/m.sup.2 weekly
Agent to be selected in consultation with FDA (i.e. gerncitabine, capecitabine, ixabepilone,vinorelbine, or carboplatin)

OR

Arm B:

Because there is no standard treatment for triple-negative metastatic breast cancer, we intend to engage the FDA in discussions and determine which combination (e.g., gemcitabine-carboplatin, ixabepilone-capecitabine, etc) is the most suitable comparator agent for the Agency.

Stratification Factors

1. Age at randomization (≧50 years versus .Itoreq.50 years)

2. Prior anthracycline use (yes versus no)

3. Geographical Area (North America versus Europe versus other)

Study Size

The target accrual is 450 randomized patients, 225 in each arm.

Study Population

Inclusion Criteria: Patients must meet all of the following criteria to be eligible for participation in the study:

1. Women 18 years of age or older.

2. Adenocarcinoma of the breast that is histologically or cytologically proven to show triple-negative markers (ER−, PR−, HER2−)

3. Metastatic disease, using the American Joint Committee on Cancer staging criteria.

4. No prior chemotherapy for metastatic disease. Prior chemotherapy in adjuvant or neo-adjuvant setting is allowed if completed at least 1 year earlier. Prior adjuvant or neo-adjuvant chemotherapy within 12 months is allowed, if completed >4 weeks previously and if it did NOT include anthracyclines or taxanes or prior trastuzumah. Patients must not have received a cumulative dose of doxorubicin of greater than 300 mg/m² or epirubicin of greater than 600 mg/m².

5. Prior hormonal therapy is allowed in either the metastatic or adjuvant setting, but must be discontinued prior to first study drug administration.

6. At least one lesion that is measurable in one dimension (RECIST). Patients may have non-measurable disease as long as they have at least one measurable lesion.

7. Eastern Cooperative Oncology Group (ECOG) performance status 0-1 (Appendix B) and an anticipated life expectancy of .gtoreq.6 months.

8. Adequate bone marrow function: i. Absolute neutrophil count (ANC) >2,000/mm³ ii. Platelet count >100,000/mm³ iii. Hemoglobin >10 g/D1

9. Adequate liver and kidney function: i. Total bilirubin within normal limits fix the institution ii. Aspartate aminotransferase (AST, SGOT) <3× the upper limit normal (ULN) (or ≦5× ULN in presence of metastatic liver disease) iii. Alanine aminotransferase (ALT, SGPT) <3× ULN (or ≦5× ULN in presence of metastatic liver disease) iv. Serum creatinine <2.0 mg/dL Alkaline Phosphatase <3× ULN in the absence of bone metastases. If Alkaline Phosphatase >3× ULN in the presence of metastatic liver disease, or attributable to bone metastases, patients will be eligible.

10. Left ventricular ejection fraction (LVEF) within institutional normal range measured by MUGA or echocardiogram with MUGA being the preferred method. Note: with either method, the results will be centrally reviewed in a blinded fashion. Eligibility is based on site's initial reading.

11. Negative serum or urine p-hCG pregnancy test for women of childbearing potential within 7 days of study drug administration (i.e., women who are not surgically sterile or more than 2 years post-menopausal).

12. Adequate birth control measures by women of childbearing potential to prevent pregnancy during the study.

13. At least 4 weeks since major surgery, 3 weeks since radiotherapy, and discontinued hormone therapy (tamoxifen or aromatase inhibitors).

14. Written, signed and dated, informed consent. Note: Informed consent must be Obtained at the time of patient screening, and before any procedures specifically related to this study are performed.

Exclusion Criteria: Patients meeting any of the following criteria are ineligible for participation in the study:

1. Prior chemotherapy or other systemic therapy other than hormonal therapy for metastatic disease.

2. Active, unresolved infection.

3. Prior adjuvant therapy with doxorubicin >300 mg/m² or epirubicin >600 mg/m².

4. Patients who develop metastatic disease 12 months after completing adjuvant paclitaxel, docetaxel, or doxorubicin/epirubicin are considered to have had prior therapy for metastatic disease and are excluded from study participation.

5. Receiving concurrent hormonal therapy.

6. Prior radiation therapy ending less than three weeks before the start of study therapy.

7. Prior radiation therapy to the mediastinal area >3,500 cGy, or radiation to >25% of the bone marrow.

8. Symptomatic brain metastases, including patients requiring corticosteriod treatment or anti-convulsant medications for control of symptoms.

9. Active cardiac disease: i. Any prior myocardial infarction. ii. Current or history of documented congestive heart failure (CHF). iii. Current use of digitalis glycosides or ACE inhibitors for CHF, Note: ACE inhibitors can he used for hypertension. iv. Any prior history of arrhythmia or cardiac valvular disease requiring medications or considered clinically significant (e.g., under the care of a cardiologist). v. Current use of medications for treatment of arrhythmias or angina pectoris. vi. Current uncontrolled hypertension (diastolic >100 mmHg or systolic >200 mmHg). vii. Clinically significant pericardial effusion.

10. Prior malignancy within 5 years, except carcinoma in situ of the cervix or non-melanoma skin cancer.

11. Women who are pregnant or breast feeding.

12. Women of childbearing potential or sexual partner unwilling to employ adequate contraception.

13. History of hypersensitivity reaction to anthracyclines, benzyl alcohol, G-CSF, Cremophor, eggs, or egg products.

14. Investigational agent(s) within 3 weeks of start of study therapy.

15. Known HIV infection.

16. Receiving any other standard or investigational treatment for cancer, or any other investigational agent for any indication.

17. Any concurrent medical or psychological condition that would limit the ability of the patient to provide informed consent or to comply with the obligations of the study.

The patents and publications listed herein describe the general skill in the art and are hereby incorporated by reference in their entireties for all purposes and to the same extent as if each was specifically and individually indicated to be incorporated by reference. In the case of an conflict between a cited reference and this specification, the specification shall control. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

1. A method for treating triple-negative metastatic breast cancer comprising administering to an individual in need thereof a dosing regimen which comprises nonpegylated liposomal doxorubicin and paclitaxel.

2. A method according to claim 1, wherein said individual previously has been administered an anthracycline.

3. A method according to claim 1, wherein the dosing regimen further comprises at least one other agent selected from the group consisting of capecitabine, carboplatin, vinorelbine, and gemcitibine.

4. A method according to claim 1, wherein said dosing regimen comprises at least one 3-week long treatment cycle.

5. A method according to claim 4, wherein the dosing regimen comprises nonpegylated liposomal doxorubicin at a dose level of from 30 mg/m² to 75 mg/m² on day 1 of each treatment cycle.

6. A method according to claim 4, wherein the dosing regimen comprises paclitaxel at a dose level of from 50 mg/m²to 250mg/m² on day 1 of each treatment cycle.

7. A method according to claim 3, wherein the at least one other agent selected from the group consisting of capecitabine, carboplatin, ixabepilone, vinorelbine, and gemcitihine is administered on day 1 of the first day of each treatment cycle.

8. A method according to claim 1, wherein the individual is not administered trastuzumab.

9. A method according to claim 2, wherein said previous administration of anthracycline was for the treatment of cancer.

10. A method according to claim 9, wherein said previous administration of anthracycline was for the treatment of breast cancer.

11. A method according to claim 2, wherein said anthracycline previously administered to said individual is selected from the group consisting of doxorubicin, idarubicin, epirubicin and daunorubicin.

12. A method according to claim 11, wherein said anthracycline previously administered to said individual is doxorubicin.

13. A method according to claim 2, wherein the total amount of said anthracycline previously administered to said individual is from 7 mg/m² to 450 mg/m².

14. A method according to claim 13, wherein the total amount of said anthracycline previously administered to said individual is from 100 mg/m² to 400 mg/m².

15. A method according to claim 1, wherein said dosing regimen does not substantially increase the likelihood that said individual will develop palmar-plantar erythrodysesthesia during said dosing regimen.

16. A method according to claim 1, wherein said dosing regimen does not substantially increase the likelihood that said individual will develop congestive heart failure during said dosing regimen.

17. A method according to claim 1, wherein said dosing regimen does not substantially increase the likelihood that said individual will suffer cardiac death during said dosing regimen.

18. A method according to claim 4, wherein said dosing regimen comprises six consecutive 3-week long treatment cycles.

19. A method according to claim 18, wherein said nonpegylated liposomal doxorubicin is administered at a dose level of about 50 mg/m² doxorubicin on day 1 of each treatment cycle, said paclitaxel is administered at a dose level of about 80 mg/m² on day 1 of the first treatment cycle and every week thereafter or at a dose level of about 75 mg/m² on day 1 of each treatment cycle or at a dose level of from 200 mg/m² to 250 mg/m² on day 1 of each treatment cycle, and said additional agent(s) is administered according to the FDA approved dosing guidelines for said agents) on day 1 of the first treatment cycle and subsequently according to the recommended schedule for the agent selected.

20. A method according to claim 19, wherein said paclitaxel is administered at a dose level of about 80 mg/m² on day 1 of the first treatment cycle and every week thereafter.
